(19) United States
(12) Patent Application Publication
(10) Pub. No.: US 2005/0085682 A1
(43) Pub. Date: Apr. 21, 2005

(54) Method and apparatus for dispensing radioactive liquid

(76) Inventors: Motoji Sasaki, Tokyo (JP); Akira Tanaka, Tokyo (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/689,051

(22) Filed: Oct. 21, 2003

(51) Int. Cl. A61M 36/06

(52) U.S. Cl. 600/4; 222/52

(57) ABSTRACT

A method and apparatus which frees the operator from dispensing operations with a reduction in exposure. A necessary amount of radioactive drug solution is dispensed by measuring radioactivity concentration of the radioactive drug solution passing through a tube constituting the channel of the radioactive drug solution, and controlling the amount of dispensation.

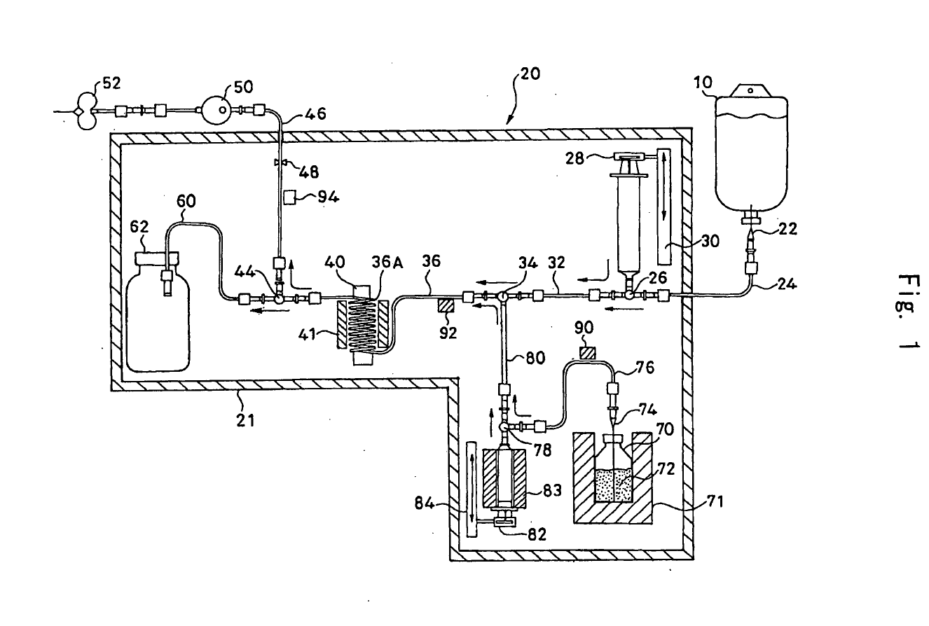

### BACKGROUND OF THE INVENTION

1. Field of the Invention

The present invention relates to a method and apparatus for dispensing a radioactive liquid. In particular, the invention relates to a method and apparatus for dispensing a radioactive liquid which are suitably applicable to the administration of a radioactive drug labeled with a radionuclide of short half-life, thus reducing operator exposure and facilitating repetitive administration, and a method and apparatus for dispensation and administration using the same.

2. Description of the Related Art

Administration of radioactive drugs labeled with highly radioactive nuclides of short half-lives to subjects in an examination room of a hospital requires a mechanism which administers predetermined doses accurately at constant speed while preventing the operator from radiation exposure. This necessitates automated, remote operation apparatuses. MR contrast medium injection systems and automatic radioactive drug injection systems have thus been put to practical use as apparatuses for administering radiation drugs to a subject automatically.

These injection systems basically comprise such components as: a syringe filled with a certain amount of drug solution; a tube leading to a subject; a final force-injection syringe for filling the tube with distilled water or physiological saline for injection, and administering the entire amount of drug solution; automatic or manual valves for switching liquid flows; an operating mechanism for constant-speed administration; and a controller.

Take, for example, the case where such an injection system is used to administer a radioactive drug (e.g., ¹⁸F-FDG (fluorodeoxyglucose), ¹³N-ammonia, ¹¹C-methionine, or the like) labeled with a nuclide of short half-life (e.g., positron emitting nuclides of ¹⁵O, ¹³N, ¹¹C, and ¹⁸F having half-lives of 2, 10, 20, and 110 minutes, respectively) to a subject. The dose of radioactivity to the subject has conventionally been determined by: loading the syringe with a solution that is prepared to a predetermined dosage of radioactivity and volume for a single person out of a large amount of radioactive drug; measuring the syringe for the amount of radioactivity before administration; administering the same to the patient manually or automatically; measuring the syringe again for the amount of radioactivity remaining; and then correcting the amount of radioactivity for attenuation with the administration time (reference time).

Here, the dispensation is desirably effected by automated remote operations in terms of preventing the operator from radiation exposure. Many apparatuses for dispensing a liquid automatically are commercially available, but have sterilization problems. In addition, the time-based attenuation of the radioactivity must be calculated with intricate operations.

Incidentally, in the apparatuses composed of sterilized equipment for automatically dispensing drugs labeled with nuclides of short half-lives, the amount of radioactivity is read directly so that a certain amount and certain concentration of radioactivity can be dispensed into the syringe and administered along with physiological saline for dilution and determination. Then, this dispensed syringe has been conventionally loaded to an administration apparatus having no dispensing mechanism as has been proposed by the applicant in Japanese Patent Laid-Open Publication No. 2000-350783. The reason is that the radioactive drugs may otherwise be wasted due to dead volumes on the channel.

According to this method, however, dispensed syringes for respective patients must be contained and transported in a lead container one by one before administered to the patients or loaded to the apparatus. This has contributed to increased exposure of the operator.

### SUMMARY OF THE INVENTION

The present invention has been achieved to solve the conventional problems described above. It is thus a first object of the present invention to free the operator from the dispensing operations to reduce radiation exposure.

A second object of the present invention is to integrate the dispensation mechanism and the administration mechanism so that repetitive administration can be performed with ease and accuracy.

The foregoing first object of the present invention has been achieved by dispensing a necessary amount of a radioactive liquid through the steps of: measuring radioactivity concentration of the radioactive liquid, for example, passing through a tube constituting a channel of the radioactive liquid; and controlling an amount of dispensation.

The foregoing first object of the present invention has also been achieved by the provision of an apparatus for dispensing a radioactive liquid for dispensing a necessary amount of radioactive liquid, the apparatus comprising: a first detector measuring radioactivity concentration of the radioactive liquid, for example, passing through a tube constituting a channel of the radioactive liquid; and a controller controlling an amount of dispensation based on the measurement.

The foregoing second object of the present invention has been achieved by the provision of a method for dispensing and administering a radioactive liquid for dispensing a necessary amount of radioactive liquid from a container and administering the same, the method comprising the steps of: dispensing the necessary amount from the container by using the foregoing dispensing method immediately before administration; holding the entire amount of the radioactive liquid just dispensed temporarily in a radiation-shielded liquid holding part; measuring an amount of radioactivity of the radioactive liquid held in the liquid holding part; and administering the entire amount of radioactive liquid.

The foregoing second object of the present invention has also been achieved by the provision of an apparatus for dispensing and administering a radioactive liquid for dispensing a necessary amount of radioactive liquid from a container and administering the same, the apparatus comprising: the foregoing dispensing apparatus for dispensing the necessary amount from the container immediately before administration; a liquid holding part capable of holding the entire amount of radioactive liquid just dispensed temporarily; a second detector measuring an amount of radioactivity of the radioactive liquid held in the liquid holding part; and administering device for administering the entire amount of radioactive liquid after the radioactivity measurement.

Furthermore, the channel of the radioactive liquid may be provided with a third detector detecting attenuation in the radioactivity concentration of the radioactive liquid and/or a passage of the radioactive liquid. This third detector can be used to minimize the amount of the radioactive liquid to be wasted in expelling air from the channel.

According to the present invention, it is possible to free the operator from the dispensing operation with a reduction in exposure. It is also possible to measure doses accurately and perform repetitive administration with ease and accuracy. Moreover, it is possible to minimize the amount of the radioactive drug solution to be wasted during dispensation.

The above and other novel features and advantages of the present invention are described in or will become apparent from the following detailed description of preferred embodiments.

### BRIEF DESCRIPTION OF THE DRAWING

The preferred embodiments will be described with reference to the drawing, wherein like elements have been denoted throughout the figures with like reference numerals, and wherein:

The drawing is a piping diagram showing the system of an embodiment of the present invention.

### DETAILED DESCRIPTION OF THE PREFERRED EMBODIMENT

Hereinafter, an embodiment of the present invention will be described in detail with reference to the drawing.

As shown in the drawing, the present embodiment is based on an administration apparatus 20 as has been proposed in Japanese Patent Laid-Open Publication No. 2000-350783, comprising an extension tube (hereinafter, referred to simply as tube) 24, a disposable physiological saline syringe for force injection (hereinafter, referred to simply as syringe) 28, a three-way cocked valve (hereinafter, referred to simply as three-way cock) 34, a radiation meter (a second detector) 40, a three-way cock 44, a tube 46, a waste bottle 62, and a controller (not shown).

In addition, the administration apparatus 20 according to the present invention further comprises a tube 76, a drug solutoin syringe 82, a radioactivity concentration sensor (a first detector) 90, and radiation pass sensors (third detectors) 92 and 94.

The tube 24 extracts physiological saline or the like for dilution from a physiological saline bag 10 which is filled with physiological saline (or distilled water for injection). The tube 24 is sterilized, and has an injection needle 22 on its extremity.

The syringe 28 injects the physiological saline or the like in the tube 24 into a tube 32 through a three-way cock 26. The syringe 28 is provided with a syringe drive 30 comprising a pulse motor, for example.

The three-way cock 26 is connected with the three-way cock 34 through the tube 32. The three-way cock 34 injects the radioactive liquid into a tube 36.

The radiation meter 40 measures the amount of radioactivity of the radioactive drug solution that is held, for example, in a buffer loop 36A of coil shape, for example. The buffer loop 36A is formed in the middle of the tube 36 and can temporarily hold the entire amount of radioactive drug solution before injection.

The three-way cock 44 switches the radioactive drug solution that is measured for the amount of radioactivity by the radiation meter 40 between being injected into a patient and being discarded.

The tube 46 injects the drug branched from the three-way cock 44 into the body of the patient through a final filter 50 and a winged needle 52 which are replaceable patient by patient. A pinch valve 48 is arranged in the middle of the tube 46.

The waste bottle 62 accommodates the waste solution which is supplied through a tube 60, being switched by the three-way cock 44.

The tube 76 dispenses the radioactive drug solution from a multiple-dose container 70 which contains a large amount, such as 50 mCi/20 ml to 200 mCi/30 ml, of radioactive drug solution 72. The tube 76 has a cathelin needle 74 on its extremity.

The drug solution syringe 82 injects a necessary amount of radioactive drug solution supplied through the tube 76 into the three-way cock 34 via a three-way cock 78 and a tube 80. The drug solution syringe 82 is driven by a syringe drive 84. The radioactivity concentration sensor 90 is intended for dispensation and detection of the presence or absence of the drug solution, being arranged in the middle of the tube 76 which extends from the cathelin needle 74 to the three-way cock 78.

The radiation pass sensor 92 is intended to detect the amount of drug solution injected for air release, being arranged in the middle of the tube 36 which extends from the three-way cock 34 to the buffer loop 36A.

The radiation pass sensor 94 is intended to detect the discharge of the drug solution, being arranged in the middle of the tube 46 which extends from the three-way cock 44 to the pinch valve 48.

In the drawing, the reference numeral 21 represents a radiation shielding wall of the entire apparatus, 41 a radiation shield for shielding the area of the radiation meter 40 from exterior, 71 a radiation shielding container for the multiple-dose bottle 70 to be inserted in, and 83 a radiation shield for shielding the drug solution syringe 82. These components are made of lead or tungsten, for example.

The incorporation of the dispensing mechanism into the administration apparatus requires that the dispensed radioactive drug solution be monitored for the amount of radioactivity and volume each time. Well type ion-chamber or other radiation detectors can be used to monitor the amount of radioactivity, whereas the detectors are large in size and thus inappropriate for incorporation into the apparatus. Then, in the present embodiment, the radioactivity concentration sensor 90 is used to measure radioactivity of part of the radioactive drug solution 72 filled into the tube 76, thereby detecting the radioactivity concentration. This allows miniaturization of the detector and its incorporation into the apparatus. Incidentally, if the entire solution is measured for radioactivity, errors can easily occur in the measurement of the amount of radioactivity due to positional errors and radioactive liquids remaining in the tube, and the dispensing accuracy may be affected. In contrast, measuring the radioactivity concentration of the tube portion can eliminate the error factors so that the volume corresponding to a desired dose of radioactivity is calculated from the measurement of the radioactivity concentration for accurate dispensation.

The desired amount of radioactive drug solution dispensed is all fed into the radiation meter 40 for accurate measurement of the amount of radioactivity. Then, the entire amount of solution can be administered at constant speed (desired speed). For repetitive administration, the conventional apparatuses have required subsections of radioactivity each time at higher risk of exposure as described above. In the present invention, on the other hand, dispensation and administration can be repeated automatically with little exposure.

Hereinafter, description will be given of the operation of the embodiment.

(1) Initially, disposable parts (syringes, three-way cocks, tubes, needles, and filters) to be replaced at a frequency of once a day or so are loaded into the apparatus 20.

(2) The multiple-dose bottle 70 containing the radioactive drug solution 72 is loaded into the apparatus as accommodated in the dedicated radiation shielding container 71, an attachment to the apparatus 20.

(3) A certain amount of the radioactive drug solution 72 in the multiple-dose bottle 70 is sucked into the drug solution syringe 82 through the tube 76 and the three-way cock 78. Next, the radioactive drug solution 72 is forced through the three-way cock 78, the tube 80, and the three-way cock 34 by the drug solution syringe 82 until the radiation pass sensor 92 senses the radioactivity. The channel extending from the tube 76 to the radiation pass sensor 92 is thus filled with the radioactive drug solution, and air is expelled from the channel at the same time. Expelling air from the channel is preferable for the sake of accurate measurement of the radioactivity concentration and dispensation of exact amount, which are absolutely indispensable to the administration to human bodies in particular. Here, the radioactivity concentration sensor 90 measures the radioactivity concentration all the time so as to minimize the amount of the radioactive drug solution that is wasted to release air from the channel.

(4) Next, the channel from the three-way cock 26 to the tube 60 is filled with physiological saline by using the physiological saline syringe 28. Meanwhile, the radioactive drug solution used in (3) is discharged to the waste bottle 62. The line extending from the three-way cock 44 to the winged needle 52 is also filled with the physiological saline. Consequently, all the channels are filled with liquid, and air is expelled.

(5) Various condition settings (the dose of radioactivity, volume, administration speed) are input from the controller (not shown) in the apparatus. The necessary volume is then calculated from the radioactivity concentration that is continuously read by the radioactivity concentration sensor 90 and the setting of the dose of radioactivity. The syringes 28 and 82 are driven and the three-way cocks are switched to fill the loop 36A with the calculated volume of radioactive drug solution.

(6) The radioactive drug solution filled in the loop 36A is measured for the amount of radioactivity accurately by the radiation meter 40.

(7) The controller in the apparatus operates to force the set volume of solution at the set speed for injection.

(8) Information such as the amount of radioactivity of the injected radioactive drug solution is printed out by a not-shown printer at the same time as the administration.

(9) The foregoing operations of dispensation, measurement, and administration are repeated.

Consequently, automatic dispensation can be performed with a minimum amount of the radioactive drug solution wasted. The operator is freed from the operations of dispensing the radioactive drug solution, filling it into the syringe, measuring the syringe for radioactivity before and after, and administering the same, which contribute exposure. A profound effect of exposure reduction can thus be expected. In addition, the administration can be repeated without exposure.

In the present embodiment, the radiation from the radioactive drug solution inside the buffer loop 36A of fixed shape, arranged in the middle of the tube 36, is measured by the radiation meter 40 which is inserted in the loop 36A, allowing miniaturization. Incidentally, the radiation meter (second detector) is not limited to this configuration, but may use well type ion-chamber or other radiation meters as heretofore. The mechanisms for forcing the drug solution and physiological saline are not limited to syringes, either.

This apparatus is basically driven by an alternating-current power source. Nevertheless, when the use with a plurality of PET scanners or the like is intended, a backup battery may be provided to maintain the state of the driving pulse motors and the like while the apparatus is unplugged for easy movement from room to room.

It is apparent that the present invention is also applicable to radioactive drugs not of short life, whereas such drugs are easy to measure accurately since they require not much anti-exposure means.

Although certain preferred embodiments have been shown and described, it should be understood that many changes and modifications may be made therein without departing from the scope of the appended claims.

1. A method for dispensing a radioactive liquid, for dispensing a necessary amount of radioactive liquid, comprising the steps of:
measuring radioactivity concentration of the radioactive liquid; and
controlling an amount of dispensation.

2. The method for dispensing a radioactive liquid according to claim 1, wherein
said radioactivity concentration of the radioactive liquid is measured when the radioactive liquid pass through a tube constituting a channel of the radioactive liquid.

3. An apparatus for dispensing a radioactive liquid for dispensing a necessary amount of radioactive liquid, the apparatus comprising:
a first detector measuring radioactivity concentration of the radioactive liquid; and
a controller controlling an amount of dispensation based on the measurement.

4. The apparatus for dispensing a radioactive liquid according to claim 3, wherein
said first detector measures the radioactivity concentration of the radioactive liquid passing through a tube constituting a channel of the radioactive liquid.

5. A method for dispensing and administering a radioactive liquid for dispensing a necessary amount of radioactive liquid from a container and administering the same, the method comprising the steps of:
dispensing the necessary amount from the container by using the dispensing method according to claim 1 or 2, immediately before administration;
holding the entire amount of the radioactive liquid just dispensed temporarily in a radiation-shielded liquid holding part;
measuring an amount of radioactivity of the radioactive liquid held in the liquid holding part; and
administering the entire amount of radioactive liquid.

6. An apparatus for dispensing and administering a radioactive liquid for dispensing a necessary amount of radioactive liquid from a container and administering the same, the apparatus comprising:
the dispensing apparatus according to claim 3 or 4, for dispensing the necessary amount from the container immediately before administration;
a liquid holding part capable of holding the entire amount of radioactive liquid just dispensed temporarily;
a second detector measuring an amount of radioactivity of the radioactive liquid held in the liquid holding part; and
administering device administering the entire amount of radioactive liquid after the radioactivity measurement.

7. The apparatus for dispensing and administering a radioactive liquid according to claim 6,
further comprising a third detector detecting attenuation in the radioactivity concentration of the radioactive liquid and/or a passage of the radioactive liquid, provided to the channel of the radioactive liquid.
